(19) United States
(12) Patent Application Publication
(10) Pub. No.: US 2016/0058634 A1
(43) Pub. Date: Mar. 3, 2016

(54) RIGID REMOVABLE DRESSING

(71) Applicants: Monty WAGNER, Tracy, CA (US); Robert J. McCUNE, Escalon, CA (US)

(72) Inventors: Monty WAGNER, Tracy, CA (US); Robert J. McCUNE, Escalon, CA (US)

(73) Assignee: TOP SHELF MANUFACTURING, LLC, TRACY, CA (US)

(21) Appl. No.: 14/839,451

(22) Filed: Aug. 28, 2015

Related U.S. Application Data
(60) Provisional application No. 62/044,860, filed on Sep. 2, 2014.

(51) Int. Cl. A61F 15/00; A61F 13/06; A61F 13/04

(52) U.S. Cl. CPC A61F 15/008; A61F 13/046; A61F 13/048; A61F 13/06; A61F 15/006; A61F 15/004

(57) ABSTRACT

A postoperative transtibial amputee protection dressing for a distal stump comprising a first rigid brace having a semi-cylindrical hollow shape including a first distal end and a first proximal end, the first distal end having a closed-shape designed to essentially match the contour of the distal stump; a second rigid brace having a semi-cylindrical hollow shape including a second distal end and a second proximal end, the second distal end having an inner contour complementary to an external contour of the first brace; and a removable flexible sleeve located at the first proximal end adapted to provide a cushioned surface at a thigh portion of the amputee, creating a barrier between the first rigid brace and the thigh portion.

### CROSS-REFERENCE TO RELATED APPLICATIONS

This application claims the benefit of U.S. Provisional Application No. 62/044,860, filed Sep. 2, 2014, the disclosure of which is herein incorporated by reference.

### BACKGROUND OF THE INVENTION

1. Field of the Invention

The present invention is directed to a rigid removable dressing adapted to accommodate transtibial amputees.

2. Description of Related Art

After an amputation of a lower limb, and specifically a transtibial (below knee) amputation, the lower limb needs to heal before a prosthesis can be fitted on the patient. Currently, about 50% of patients fall within the first 48 hours after amputation. Therefore, it is important that the lower limb is protected from falls and impacts as well as providing protection from a flexion contracture of the residual limb. Furthermore, bandages get soiled easy and need to be changed often. Consequently, there is a need for a rigid removable dressing to accommodate a transtibial amputation providing circumferential protection from falls and impacts.

### BRIEF SUMMARY OF THE INVENTION

In one embodiment of the present invention a postoperative transtibial amputee protection dressing for a distal stump comprising a first rigid brace having a semi-cylindrical hollow shape including a first distal end and a first proximal end, the first distal end having a closed-shape designed to essentially match the contour of the distal stump; a second rigid brace having a semi-cylindrical hollow shape including a second distal end and a second proximal end, the second distal end having an inner contour complementary to an external contour of the first brace; and a removable flexible sleeve located at the first proximal end adapted to provide a cushioned surface at a thigh portion of the amputee, creating a barrier between the first rigid brace and the thigh portion.

In one embodiment, a pair of relief cuts located at opposite sides of the first distal end designed provide flexibility to the distal end when additional space is required to accommodate bandages covering the distal stump is provided. In another embodiment, the second rigid brace comprises a flared crown portion having an elevated center and curved opening that is designed to prevent friction and minimize pressure and irritation to the amputee's kneecap and tibia during use. In yet another embodiment, a plurality of circular air vents located on the first and second rigid braces is provided.

In one embodiment, an adjustment screw is provided; wherein the second rigid brace comprises an adjustment tab protruding from the second distal end, the adjustment tab having a plurality of sizing holes; the first rigid brace comprising a receiving hole located at the center of the first distal end; the adjustment screw entering one of the plurality of sizing holes and the receiving hole defining a girth between the first rigid brace and the second rigid brace. In another embodiment, the adjustment tab and adjustment screw allows the second rigid brace to rotate both laterally and longitudinally in relation to the first rigid brace. The adjustment tab and adjustment screw allows the second rigid brace to rotate both laterally and longitudinally in relation to the first rigid brace.

In one embodiment, a number of stackable crescent shaped pads or pads of various thickness are positioned on an inside surface of the removable flexible sleeve at a location corresponding to the supracondylar portion of the amputee's knee joint is provided; the number of stackable crescent shaped pads are attached to the inside surface by a hook-and-loop closure system. In another embodiment, the first and second rigid braces are secured in a closed position by a pair of lower straps, and the removable flexible sleeve is secured to the thigh portion by a pair of upper straps. In yet another embodiment, a plurality of foam disks removably positioned at the first distal end providing support and protection to the distal stump is provided. In one embodiment, the removable flexible sleeve is made of neoprene.

In one embodiment, an optional waist belt having a belt portion and a suspension portion is provided, the suspension portion extending perpendicularly from the belt portion, the suspension portion designed to attach to an inelastic loop and hook system located at the second proximal end such that: the optional waist belt provides support and aids in suspension of the device while ambulating. In another embodiment, a plurality of hook-and-loop fastening pads attached to an external surface of the first proximal end is provided; wherein the removable flexible sleeve is removably attached to the first proximal end by the plurality of hook-and-loop fastening pads.

In another aspect to the invention, a method providing postoperative transtibial amputee protection for a distal stump comprising steps (a) providing a rigid removable dressing comprising a first rigid brace having a semi-cylindrical hollow shape including a first distal end and a first proximal end, the first distal end having a closed-shape designed to essentially match the contour of the distal stump; a second rigid brace having a semi-cylindrical hollow shape including a second distal end and a second proximal end, the second distal end having an inner contour complementary to an external contour of the first brace; a removable sleeve located at the first proximal end, the removable sleeve made of a stretch material; a pair of lower straps having a first hook-and-loop closure system attached to the of first rigid brace; a pair of attachment rings attached to the first rigid brace; a pair of upper straps attached to the removable sleeve; a number of stackable crescent shaped pads; and a plurality of foam disks; (b) positioning the plurality of foam disks at the first distal end; (c) inserting the distal stump into the first distal end; (d) attaching the second rigid brace to the first rigid brace by feeding the pair of lower straps through the pair of attachment rings via the first hook-and-loop closure system; (e) positioning the number of stackable crescent shaped pads on a side surface of the removable sleeve at a location corresponding to the supracondylar portion of the amputee's knee joint; and (f) securing the rigid removable dressing via the pair of upper straps, wherein the removable sleeve provides a cushioned surface at a thigh portion of the amputee, creating a barrier between the first rigid brace and the thigh portion.

In one embodiment, step (a), the first rigid brace comprises a pair of relief cuts located at opposite sides of the first distal end designed provide flexibility to the distal end when additional space is required to accommodate bandages covering the distal stump. In another embodiment, in step (a), the first and second brace comprising a plurality of circular air vents. In yet another embodiment, in step (a), the second rigid brace comprises a flared crown portion having a elevated center and curved opening that is designed to prevent friction and minimize pressure and irritation to the amputee's kneecap and tibia during use. In one embodiment, in step (d), a girth between the first rigid brace and the second rigid brace is adjusted via an adjustment screw, an adjustment tab, and a receiving hole.

In one embodiment, a further step is provided, securing an optional waist belt to the rigid removable dressing. In one embodiment, the optional waist belt comprises a belt portion and a suspension portion, the suspension portion extending perpendicularly from the belt portion, the suspension portion designed to attach to an inelastic loop and hook system located at the second proximal end such that: the optional waist belt provides support and aids in suspension of the device while ambulating. In another embodiment, the distal stump is located on a left limb or right limb, and the rigid removable dressing is designed to universally fit both the left limb and the right limb.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1A is a front perspective view of a rigid removable dressing according to an embodiment of the present invention.

FIG. 1B is a left side perspective view of the rigid removable dressing as shown in FIG. 1A.

FIG. 1C is a right side perspective view of the rigid removable dressing as shown in FIG. 1A.

FIG. 1D is a bottom perspective view of the rigid removable dressing as shown in FIG. 1A.

FIGS. 2-13 are perspective views illustrating the steps involved in applying the rigid removable dressing to a postoperative amputee according to some embodiments of the present invention.

### DETAILED DESCRIPTION OF THE PREFERRED EMBODIMENT

The following description is provided to enable any person skilled in the art to make and use the invention and sets forth the best modes contemplated by the inventors of carrying out their invention. Various modifications, however, will remain readily apparent to those skilled in the art, since the general principles of the present invention have been defined herein to specifically provide a transtibial rigid removable dressing with an improved design providing circumferential protection from falls or impact.

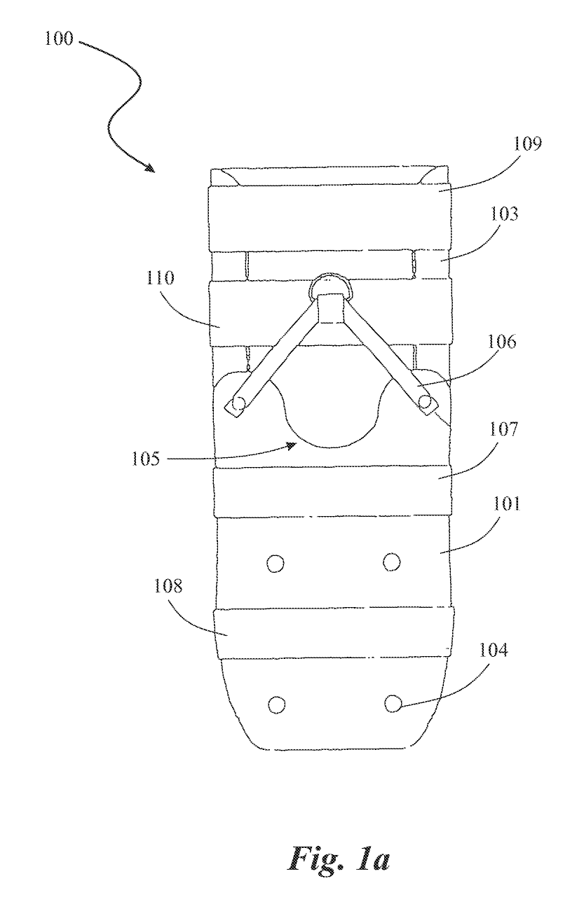

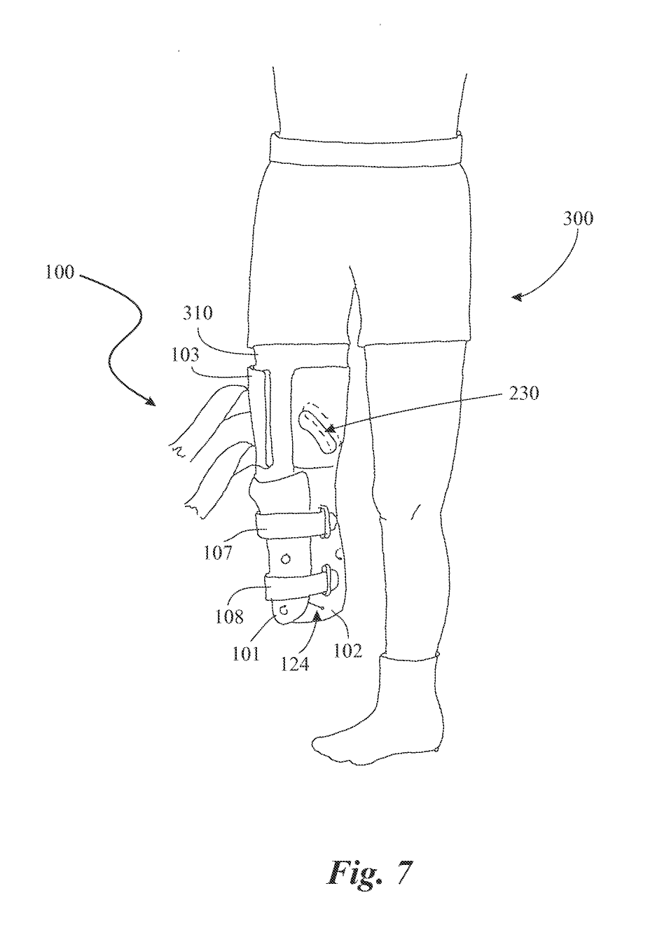

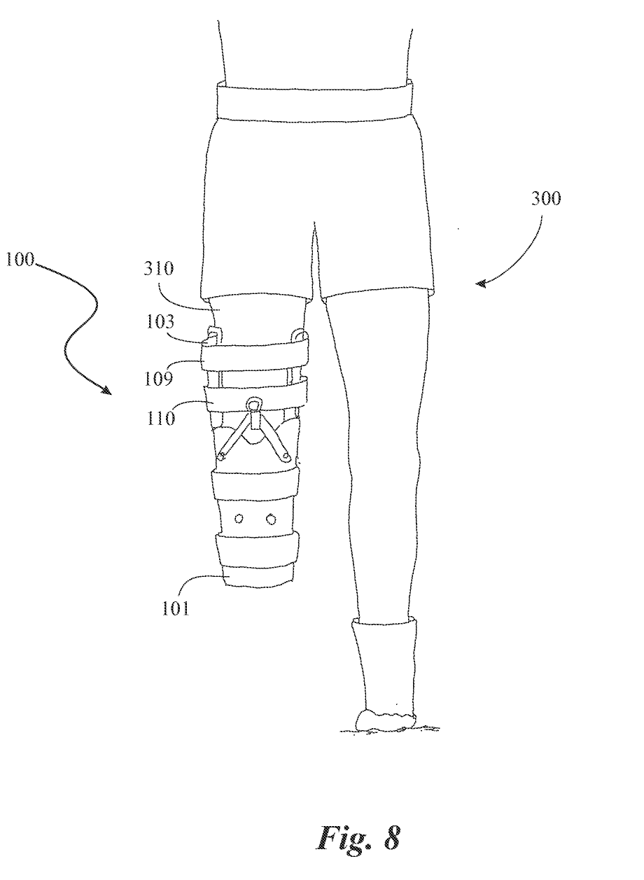

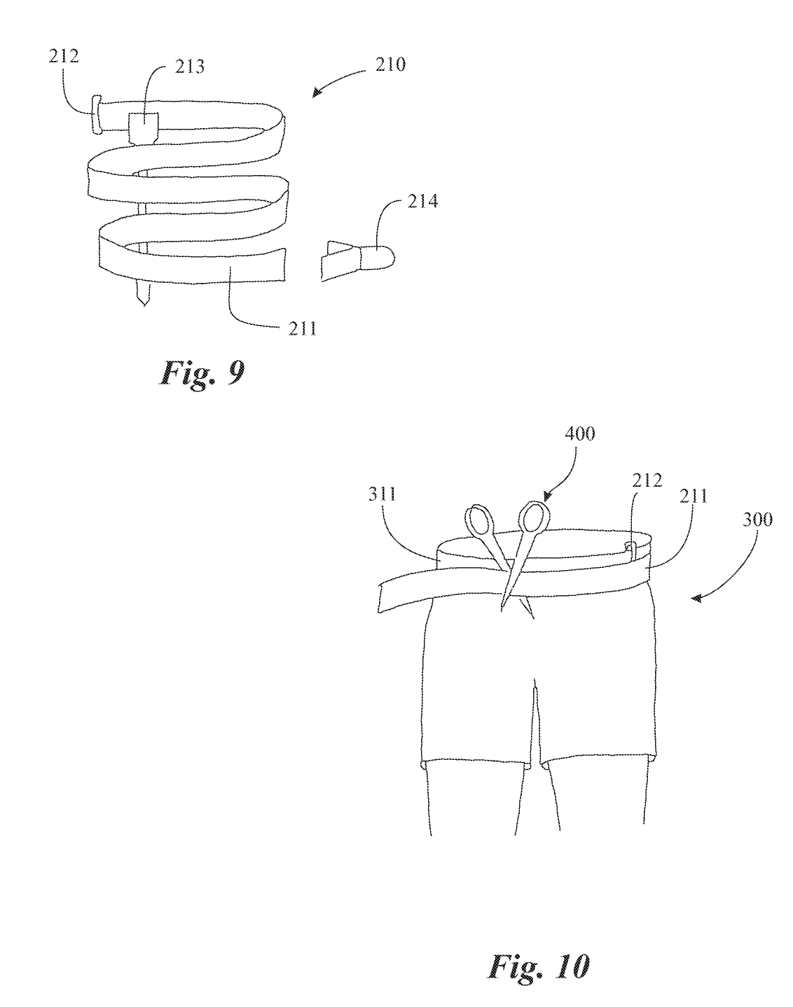

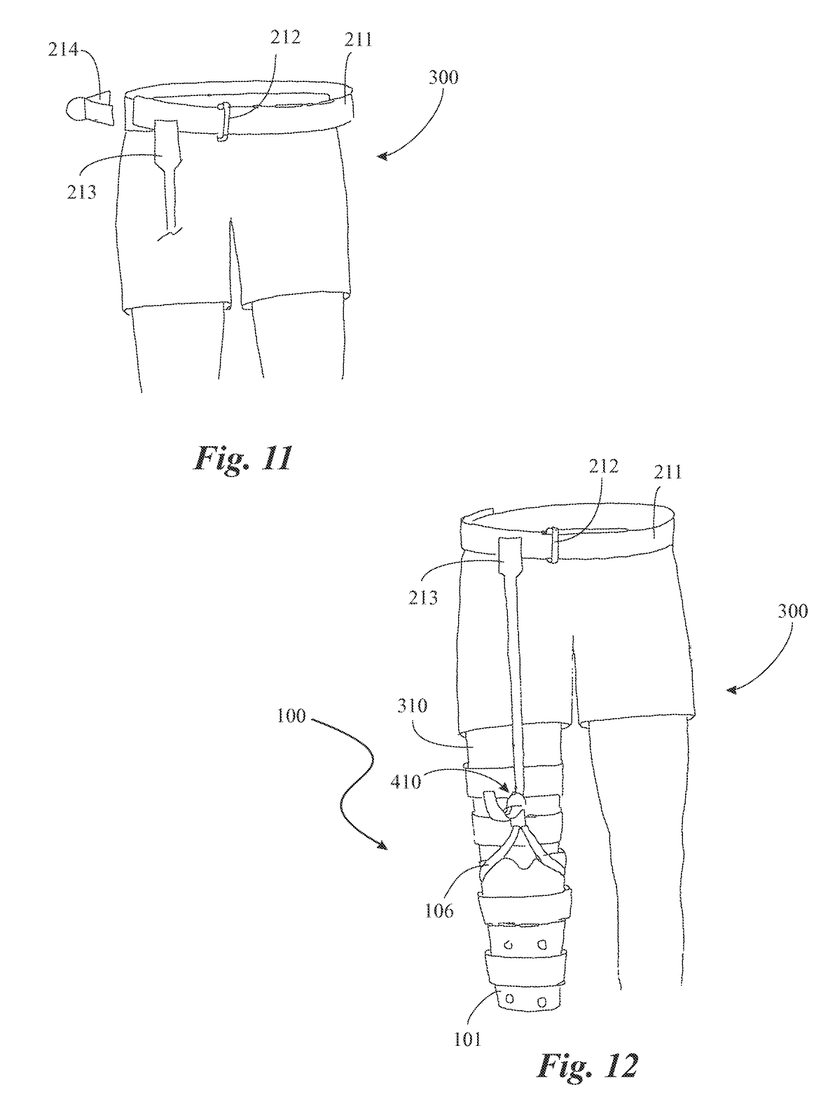

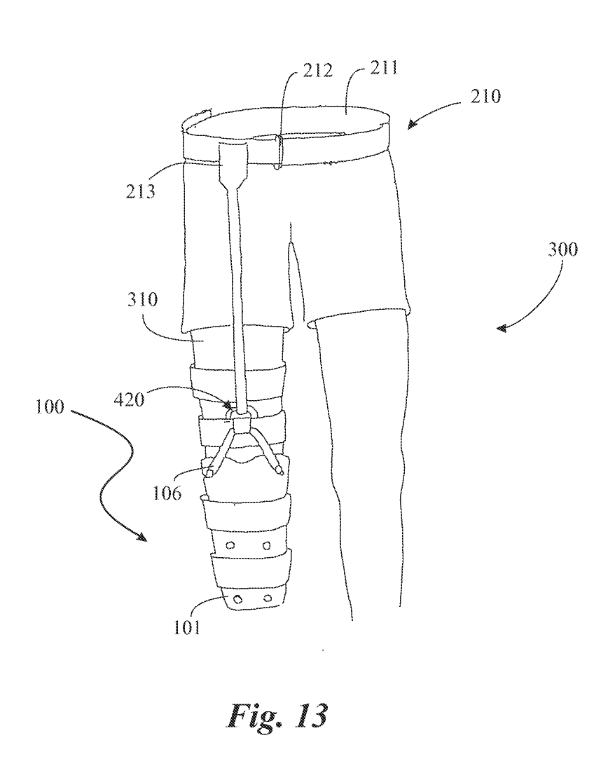

FIGS. 1 through 13 illustrate an exemplary embodiment of a rigid removable dressing 100 providing postoperative transtibial amputee protection that is rigid, adjustable, and removable. The present invention is designed be universally applied to either the left or right leg. Referring now to FIGS. 1A-1D, the rigid removable dressing comprises an anterior shell 101 and a posterior shell 102. Both the anterior and posterior shells are made from a rigid material, such as medical grade plastic. In some embodiments, the anterior and posterior shells are made from thermoplastics, and specifically a sheet thermoplastic polymer including but not limited to, low density polyethylene (PE), polypropylene (PP), or a combination thereof. The anterior shell includes a plurality of circular air vents 104 and 125 designed to provide airflow. Although the air vents are circular, it is understood that they may be any shape, and their location and position on the anterior shell may be modified. The anterior shell further includes a crown portion 105 having an elevated center of approximately 5 degrees in relation to the majority of the anterior shell. The crown portion features a flared and curved opening that is designed to prevent friction and irritation to the knee and tibia of the patient during use. An inelastic strapping 106 with an attached hook is located at the proximal end of the anterior shell. The inelastic strapping with the attached hook is used to further secure the present invention to the patient using a supplementary waist belt which will be described in greater detail below.

The posterior shell comprises a sleeve 103 located at the proximal end of the posterior shell. The sleeve is made from a soft flexible fabric, such as neoprene, to provide a cushioned, cleanable, and comfortable barrier between the rigid material of the posterior shell and the skin of the patient. Similarly to the anterior shell, the posterior shell also includes a plurality of circular air vents 111 designed to provide airflow to the patient. The posterior shell further includes a set of relief cuts 124 located on each side towards the distal end of the posterior shell. The relief cuts provide additional girth and space for bandages worn by the patient after the transtibial amputation to accommodate for swelling and variability of residual limb size.

The anterior and posterior shells are secured via lower straps 107 and 108 with hook-and-loop fastening means 119 and 120, such as Velcro®. Specifically, the lower straps are anchored to the left side of the posterior shell via rivets 118 and 119, and then looped through ring assemblies 116 and 117 located on the right side of the posterior shell. The lower straps are finally folded back and affixed to themselves with the hook-and-loop fastening means. Two upper straps 109 and 110 are attached the sleeve via attachment means 122 and 123 and secured to the sleeve with hook-and-loop patches 114 and 115, as well as clasps 112 and 113. The attachment means may including a number of attachment methods, including but not limited to hook-and-loop fasteners, welding, stitching, and lamination. The width or girth between the anterior and posterior shells may be adjusted using screw 127 and plurality of sizing holes 126. The sizing holes are located on an adjustment tab 131 protruding from the bottom of the anterior shell. The adjustment tab and screw enables the anterior shell to rotate both laterally arid longitudinally in relation to the posterior shell. The girth adjustment will be described in greater detail below.

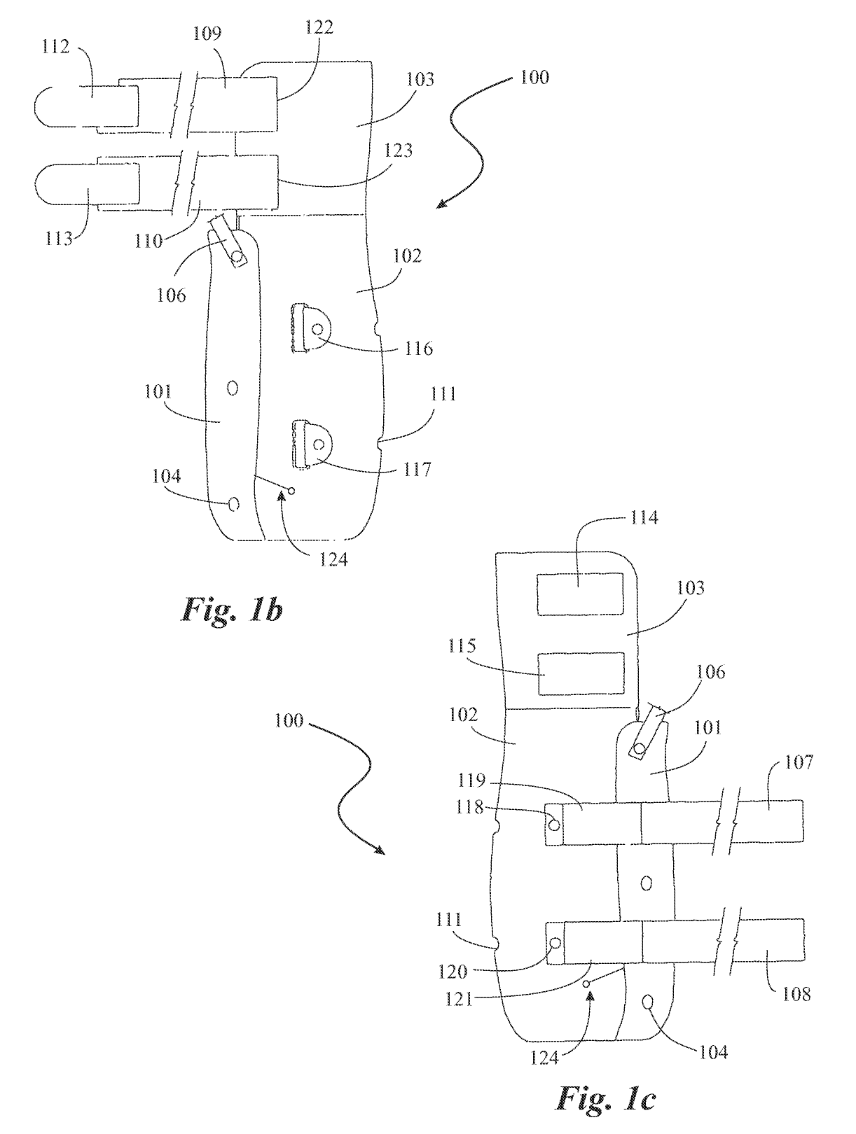

As shown in FIG. 2, the initial step involved in applying the rigid removable dressing to a postoperative amputee according to some embodiments of the present invention is to remove a supplementary waist belt 210, foam disks 220, and crescent shape pads 230 that are stored inside rigid removable dressing when provided to a patient. Next, the lower and upper straps should be disengaged to allow the present invention to be placed on the patient, as will be described below.

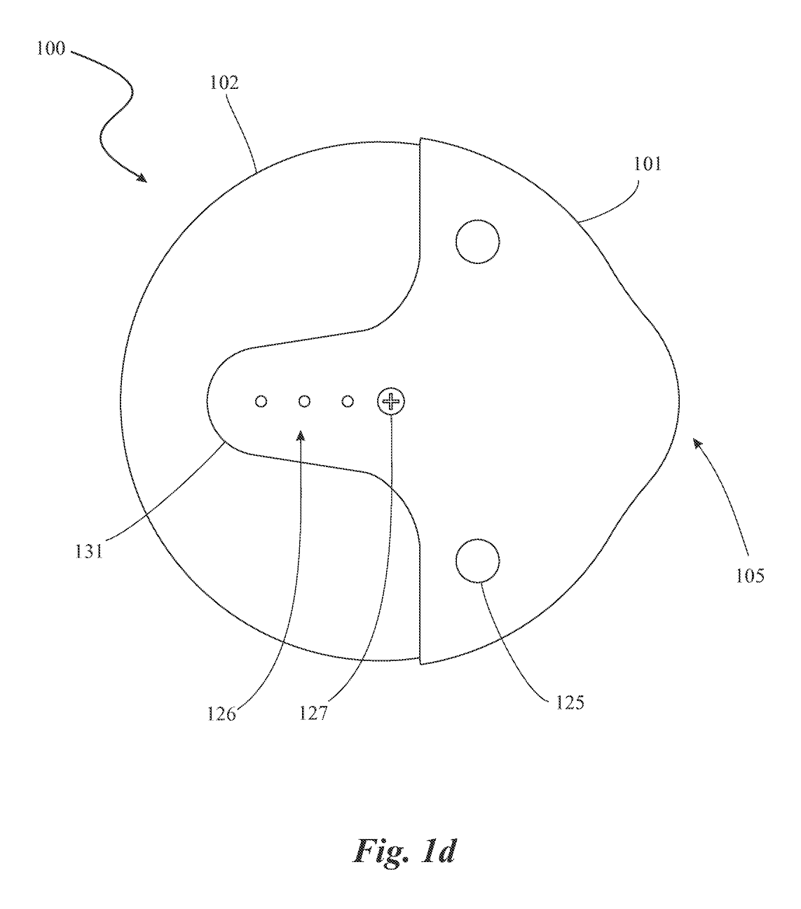

As shown in FIG. 3, the next step involved in applying the rigid removable dressing to a postoperative amputee 300 is to position the rigid removable dressing alongside the residual limb 310 to determine the approximate length and the amount of padding or foam disks 220 needed to be inserted into the distal end prior to installation. Each foam disk is circular having a width of one inch, and is designed to provide additional protection to the distal portion of the limb, as well as to accommodate the length and correct positioning of the present invention. For instance, if the residual limb is shorter than the length of the rigid removable dressing, the foam disks may be added to fill the void at the distal end of the rigid removable dressing.

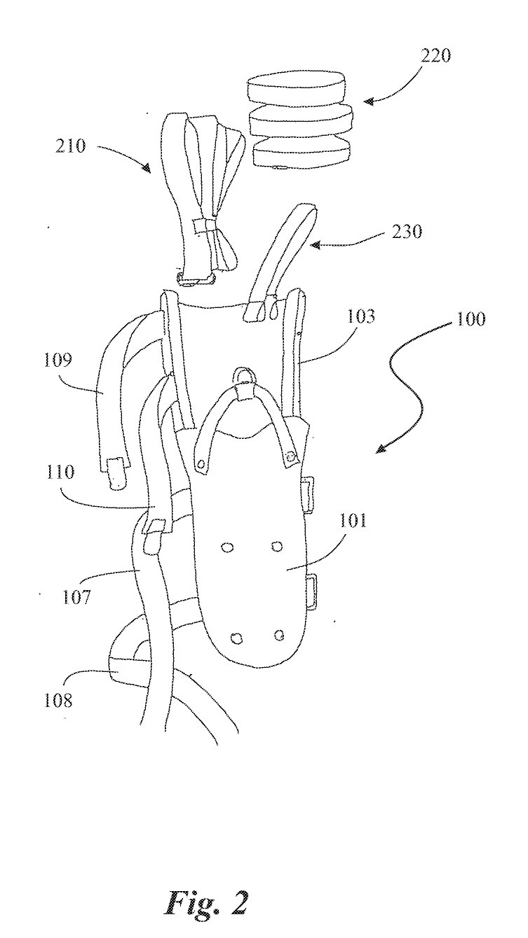

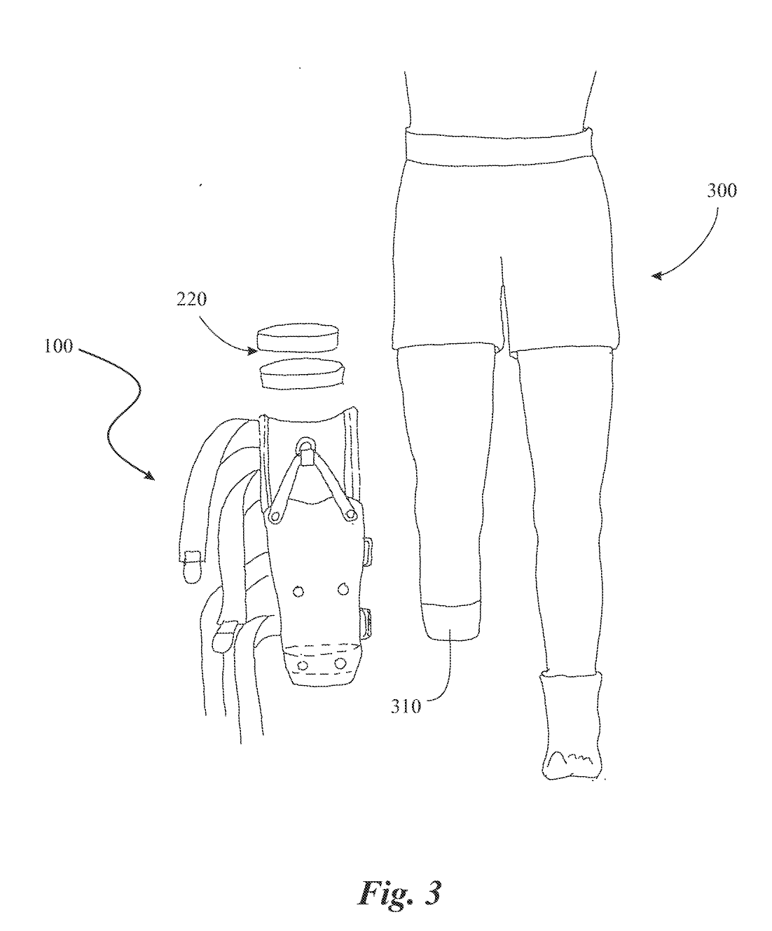

FIGS. 4-5b illustrate the next steps involved in applying the rigid removable dressing to the postoperative amputee. Specifically, the next step is to open or separate the anterior shell from the posterior shell, and slide the rigid removable dressing over the surgical dressing and residual limb 310, as shown in FIG. 4. If the surgical dressing is smaller or larger than the distal portion of the ridged removable dressing, the girth may be adjusted using screw 127 and a plurality of sizing holes 126. Each sizing hole allows for approximately one half inch adjustment, and a total of two inches in overall girth adjustment. The patient may adjust the girth or width accordingly by engaging the screw through the correct sizing hole and receiving hole 128 positioned at distal end of the posterior shell. In sonic embodiments, additional circular air vents 125 are positioned on the bottom surface of the posterior shell at the distal end. Ideally, the rigid removable dressing should be positioned so that the kneecap and tibia of the patient is protected by crown portion 105, as previously mentioned.

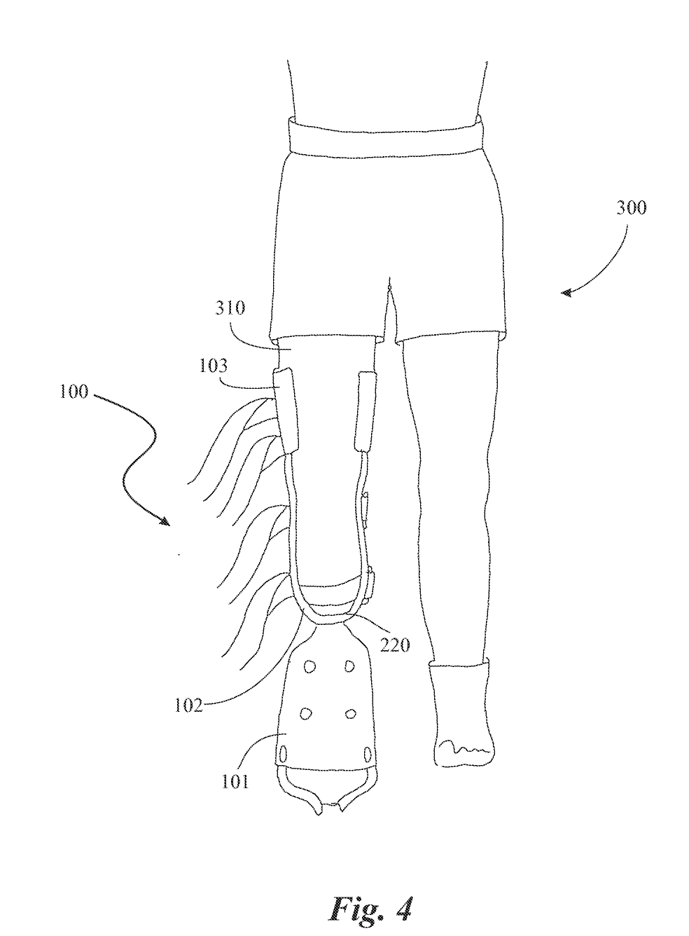

FIG. 6 illustrates the next step involved in applying the rigid removable dressing to the postoperative amputee according to some embodiments of the present invention. Lower straps 107 and 108 are feed through ring assemblies 116 and 117 respectively, and affixed using attachment means (119 and 121; FIG. 1C), securing the anterior shell to the posterior shell.

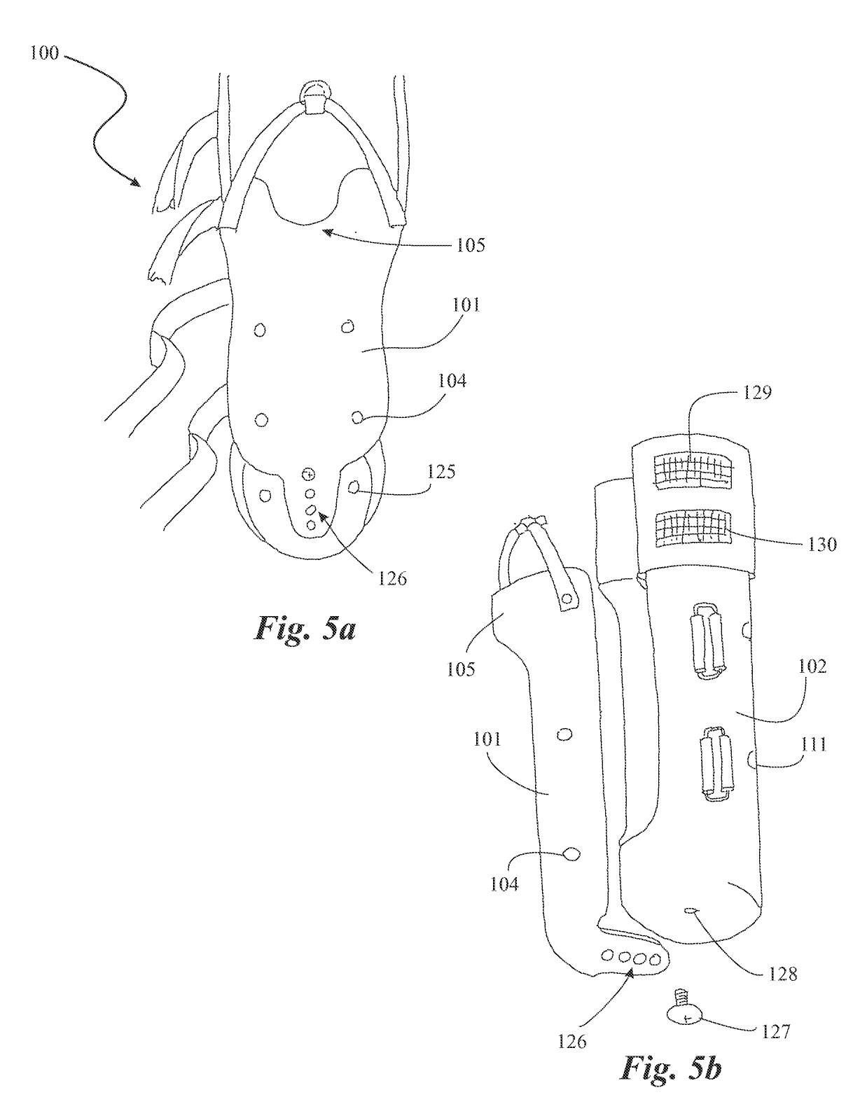

FIG. 7 illustrates the next step involved in applying the rigid removable dressing to the postoperative amputee according to some embodiments of the present invention. Crescent shaped pads 230 are positioned on the medial or lateral aspects of the knee at the supracondylar portion of the knee joint. The pads aid in the anti-migration and secure fit of the rigid removable dressing. One or more pads may be stacked for additional support and compression. Each pad has a bottom surface comprised of a hook-and-loop fastening system, such as Velcro®, for securing the pad to the inner surface of sleeve 103 as illustrated. In some embodiments, the pads are held in place with adhesive means or pockets.

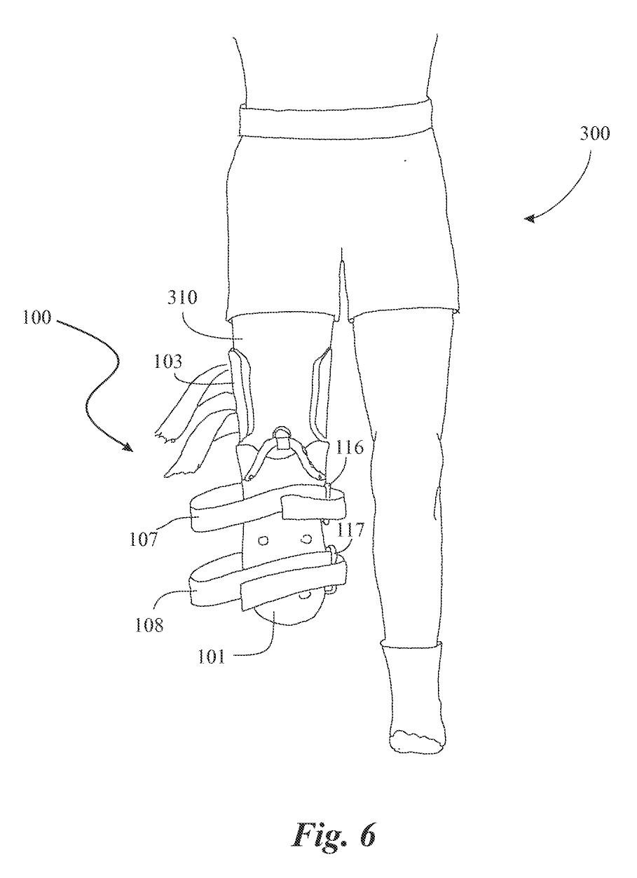

FIG. 8 illustrates the next step involved in applying the rigid removable dressing to the postoperative amputee according to some embodiments of the present invention. Upper straps 109 and 110 located on sleeve 103 are secured via hook-and-loop patches 114 and 115 (FIG. 1C) and clasps 112 and 113 (FIG. 1B). The sleeve covers the thigh portion of the patient. For a patient with a smaller circumference thigh the straps may be cut to length and re-secured. Specifically, the clasps include a “y” shaped section that can be removed from the end of the straps during resizing then re-attached for re-securement.

FIGS, 9-13 illustrate steps involved in attaching a supplementary waist belt 210 to further secure the rigid removable dressing to the postoperative amputee according to some embodiments of the present invention. The waist belt comprises belt portion 211, ring 212, attachment portion 213, and adapter clasp 214. The adapter clasp functions similarly to clasps 112 and 113 (FIG. 1B), as it includes a “y” shaped section, allowing the adapter clasp to be removed from the belt portion and reattached. In the first step, the belt portion is wrapped around the patient's midsection 311, overlapping the belt portion approximately one foot. Next, the excess length in the belt portion is cut 400, and the remaining belt portion is fed through the ring. The belt is secured around the midsection using adapter clasp 214. In the next step, attachment portion 213 is fed through 410 the attached hook of inelastic strapping 106. Last, the attachment portion is adjusted for length and secured back on itself using a clasp (not pictured) that functions similarly to the adapter clasp previously discussed. Once secured 420, the waist belt aids in the suspension of the rigid removable dressing while ambulating. When the rigid removable dressing is properly secured, the clasp material should not nm against the patient's skin. The waist belt is optional, and is not required.

After a period of time when maintenance is necessary, sleeve 103 may be removed for cleaning. The sleeve is attached to posterior shell 102 with rectangular hook-and-loop attachment elements 129 and 130 (FIG. 5), which are attached to the posterior shell with adhesive means. To prevent possible allergic reactions, the rigid removable dressing is latex free.

Although the invention has been described in considerable detail in language specific to structural features and or method acts, it is to be understood that the invention defined in the appended claims is not necessarily limited to the specific features or acts described. Rather, the specific features and acts are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit scope of the invention.

In addition, reference to “first,” “second,” “third,” and etc. members throughout the disclosure (and in particular, claims) are not used to show a serial or numerical limitation but instead are used to distinguish or identify the various members of the group.

What is claimed is:

1. A postoperative transtibial amputee protection dressing for a distal stump comprising:
a first rigid brace having a semi-cylindrical hollow shape including a first distal end and a first proximal end, the first distal end having a closed-shape designed to essentially match the contour of the distal stump;
a second rigid brace having a semi-cylindrical hollow shape including a second distal end and a second proximal end, the second distal end having an inner contour complementary to an external contour of the first brace;
a removable flexible sleeve located at the first proximal end adapted to provide a cushioned surface at a thigh portion of the amputee, creating a barrier between the first rigid brace and the thigh portion.

2. The dressing of claim 1, further comprising a pair of relief cuts located at opposite sides of the first distal end designed provide flexibility to the distal end when additional space is required to accommodate bandages covering the distal stump.

3. The dressing of claim 1, wherein the second rigid brace comprises a flared crown portion having an elevated center and curved opening that is designed to prevent friction and minimize pressure and irritation to the amputee's kneecap and tibia during use.

4. The dressing of claim 1, further comprising a plurality of circular air vents located on the first and second rigid braces.

5. The dressing of claim 1, further comprising an adjustment screw; wherein the second rigid brace comprises an adjustment tab protruding from the second distal end, the adjustment tab having a plurality of sizing holes; the first rigid brace comprising a receiving hole located at the center of the first distal end; the adjustment screw entering one of the plurality of sizing holes and the receiving hole defining a girth between the first rigid brace and the second rigid brace.

6. The dressing of claim 5, wherein the adjustment tab and adjustment screw allows the second rigid brace to rotate both laterally and longitudinally in relation to the first rigid brace.

7. The dressing of claim 1, further comprising a number of stackable crescent shaped pads positioned on an inside surface of the removable flexible sleeve at a location corresponding to the supracondylar portion of the amputee's knee joint; the number of stackable crescent shaped pads are attached to the inside surface by a hook-and-loop closure system.

8. The dressing of claim 1, wherein the first and second rigid braces are secured in a closed position by a pair of lower straps, and the removable flexible sleeve is secured to the thigh portion by a pair of upper straps.

9. The dressing of claim 1, further comprising a plurality of foam disks removably positioned at the first distal end providing support and protection to the distal stump.

10. The dressing of claim 1, wherein the removable flexible sleeve is made of neoprene.

11. The dressing of claim 1, further comprising an optional waist belt having a belt portion and a suspension portion, the suspension portion extending perpendicularly from the belt portion, the suspension portion designed to attach to an inelastic loop and hook system located at the second proximal end such that: the optional waist belt provides support and aids in suspension of the device while ambulating.

12. The dressing of claim 10, further comprising a plurality of hook-and-loop fastening pads attached to an external surface of the first proximal end; wherein the removable flexible sleeve is removably attached to the first proximal end by the plurality of hook-and-loop fastening pads.

13. A method providing postoperative transtibial amputee protection for a distal stump comprising steps:
(a) providing a rigid removable dressing comprising a first rigid brace having a semi-cylindrical hollow shape including a first distal end and a first proximal end, the first distal end having a closed-shape designed to essentially match the contour of the distal stump; a second rigid brace having a semi-cylindrical hollow shape including a second distal end and a second proximal end, the second distal end having an inner contour complementary to an external contour of the first brace; a removable sleeve located at the first proximal end; the removable sleeve made of a stretch material; a pair of lower straps having a first hook-and-loop closure system attached to the of first rigid brace; a pair of attachment rings attached to the first rigid brace; a pair of upper straps attached to the removable sleeve; a number of stackable crescent shaped pads; and a plurality of foam disks;
(b) positioning the plurality of foam disks at the first distal end;
(c) inserting the distal stump into the first distal end;
(d) attaching the second rigid brace to the first rigid brace by feeding the pair of lower straps through the pair of attachment rings via the first hook-and-loop closure system;
(e) positioning the number of stackable crescent shaped pads on an inside surface of the removable sleeve at a location corresponding to the supracondylar portion of the amputee's knee joint; and
(f) securing the rigid removable dressing via the pair of upper straps, wherein t removable sleeve provides a cushioned surface at a thigh portion of the amputee, creating a barrier between the first rigid brace and the thigh portion.

14. The method of claim 13, wherein in step (a), the first rigid brace comprises a pair of relief cuts located at opposite sides of the first distal end designed provide flexibility to the distal end when additional space is required to accommodate bandages covering the distal stump. 15, The method of claim 13, wherein in step (a), the first and second brace comprising a plurality of circular air vents.

16. The method of claim 13, wherein in step (a), the second rigid brace comprises a flared crown portion having an elevated center and curved opening that is designed to prevent friction and minimize pressure and irritation to the amputee's kneecap and tibia during use.

17. The method of claim 13, wherein in step (d), a girth between the first rigid brace and the second rigid brace is adjusted via an adjustment screw, an adjustment tab, and a receiving hole.

18. The method of claim 13, further providing a step securing an optional waist belt to the rigid removable dressing.

19. The method of claim 18, wherein the optional waist belt comprises a belt portion and a suspension portion, the suspension portion extending perpendicularly from the belt portion, the suspension portion designed to attach to an inelastic loop and hook system located at the second proximal end such that: the optional waist belt provides support and aids in suspension of the device while ambulating.

20. The method of claim 13, wherein the distal stump is located on a left limb or right limb, and the rigid removable dressing is designed to universally fit both the left limb and the right limb.
